(19) United States
(12) Patent Application Publication
(10) Pub. No.: US 2009/0163959 A1
(43) Pub. Date: Jun. 25, 2009

(54) ROTATIONAL OSTEOTOMY PLATING SYSTEM

(76) Inventors: Nick A. Deeter, Warsaw, IN (US); Gary D. Barnett, Wabash, IN (US); Michael C. Jones, North Webster, IN (US); David W. Daniels, Winona Lake, IN (US)

Correspondence Address: BINGHAM MCHALE LLP, 2700 MARKET TOWER, 10 WEST MARKET STREET, INDIANAPOLIS, IN, 46204-4900, US

(21) Appl. No.: 12/265,471

(22) Filed: Nov. 5, 2008

Related U.S. Application Data
(60) Provisional application No. 60/985,448, filed on Nov. 5, 2007.

(51) Int. Cl. A61B 17/70

(52) U.S. Cl. 606/280; 606/286

(57) ABSTRACT

A bone plating system to aid in tibial rotational osteotomy correction. Also, a method for correcting a long bone that has an angular misalignment.

### CROSS REFERENCE TO RELATED APPLICATION

This application claims priority to U.S. Provisional Patent Application Ser. No. 60/985,448, filed Nov. 5, 2007, all of which is incorporated herein by reference.

### FIELD OF THE INVENTION

Various embodiments of the present invention pertain to methods and apparatus for correction of an angular misalignment in a bone, and in particular to a system of plates that supports both sections of a cut bone in which the angle has been corrected.

### BACKGROUND OF THE INVENTION

Angular misalignment of a long bone such as a tibia can result in a permanent toeing-in position of the foot, sometimes referred to pigeon toe. This condition can be corrected by surgery. Currently, surgeons will drill two pins into the bone then cut the tibia in two. Once cut, they rotate the bone while observing the pin locations. Once the desired rotation is achieved, they couple the two pieces of bone together with a single plate that extends across the cut, with the intent of not changing the desired rotation.

### SUMMARY OF THE INVENTION

One aspect of some embodiments of the present invention pertain to a tibial plating system for use in performing rotational tibial osteotomies.

Yet another aspect of some embodiments pertain to a method that uses two primary plates that are aligned and screwed into place prior to cutting the tibia. Once the primary plates are screwed into place, the tibia is cut. This allows the surgeon to rotate the tibia to make the correction. After rotation, a secondary plate is fastened to each primary plate to maintain the correction.

Yet another aspect pertains to an orthopedic plating system that includes first and second primary plates attached to a long bone. These plates are interconnected by a secondary plate that is curved about a first axis generally parallel to the longitudinal axis of a long bone, and further curved about a second axis that is at least partly orthogonal to the first axis.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these myriad combinations is excessive and unnecessary.

### BRIEF DESCRIPTION OF THE DRAWINGS

FIG. 1 shows a representation of a method of repairing a bone according to one embodiment of the present invention.

FIG. 2 represents a succeeding part of the method begun in FIG. 1.

FIG. 3 represents a succeeding part of the method begun in FIG. 1.

FIG. 3a is a cross sectional view of the aligned bone portions as viewed from line 3a-3a of FIG. 3.

FIG. 4 is a frontal view of a secondary plate according to one embodiment of the present invention.

FIG. 5 is a view of the plate of FIG. 4 as taken along line 5-5.

FIG. 6 is a view of the plate of FIG. 4 as taken along line 6-6.

FIG. 7 shows frontal, side, and top views of a primary plate according to one embodiment of the present invention.

FIG. 8 shows frontal, side, and top views of a primary spacing device according to one embodiment of the present invention.

### DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

The use of an N-series prefix for an element number (NXX.XX) refers to an element that is the same as the non-prefixed element (XX.XX), except as shown and described thereafter. As an example, an element 1020.1 would be the same as element 20.1, except for those different features of element 1020.1 shown and described. Further, common elements and common features of related elements are drawn in the same manner in different figures, and/or use the same symbology in different figures. As such, it is not necessary to describe the features of 1020.1 and 20.1 that are the same, since these common features are apparent to a person of ordinary skill in the related field of technology. Although various specific quantities (spatial dimensions, temperatures, pressures, times, force, resistance, current, voltage, concentrations, wavelengths, frequencies, etc.) may be stated herein, such specific quantities are presented as examples only, and are not to be construed as limiting.

Various embodiments presented herein pertain to methods and apparatus for correcting an angular offset in a bone, such as a tibia or other long bone. Some embodiments include attachment and coupling plates that are sized for pediatric patients. In pediatric applications, the system of plates described herein are preferably not placed across the growth plate of the bone, so as to not interfere with bone growth in the patient. However, other embodiments of the present invention contemplate attachment of plates to the bone at any location. Further, other embodiments of the present invention are adapted and configured to couple to adult bones.

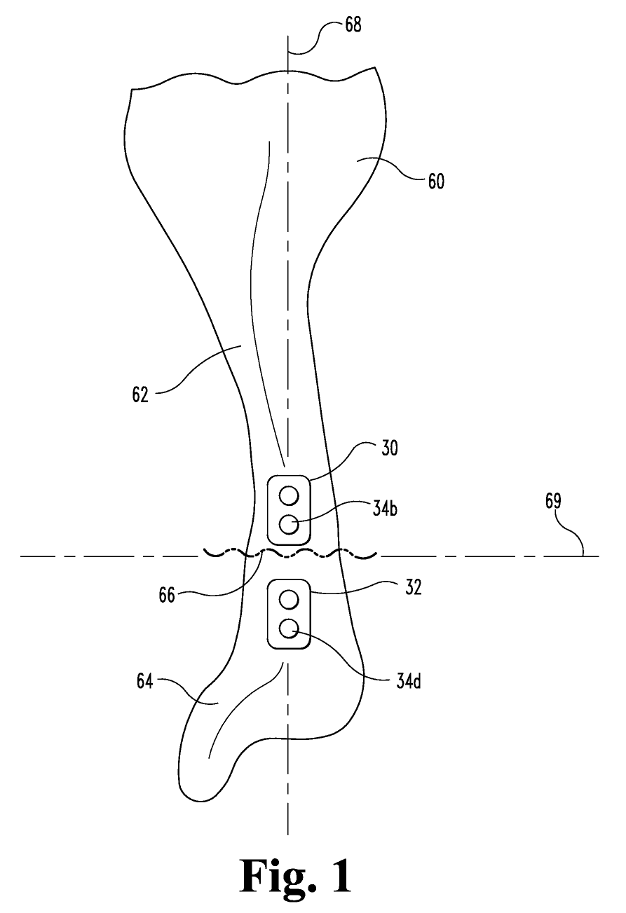

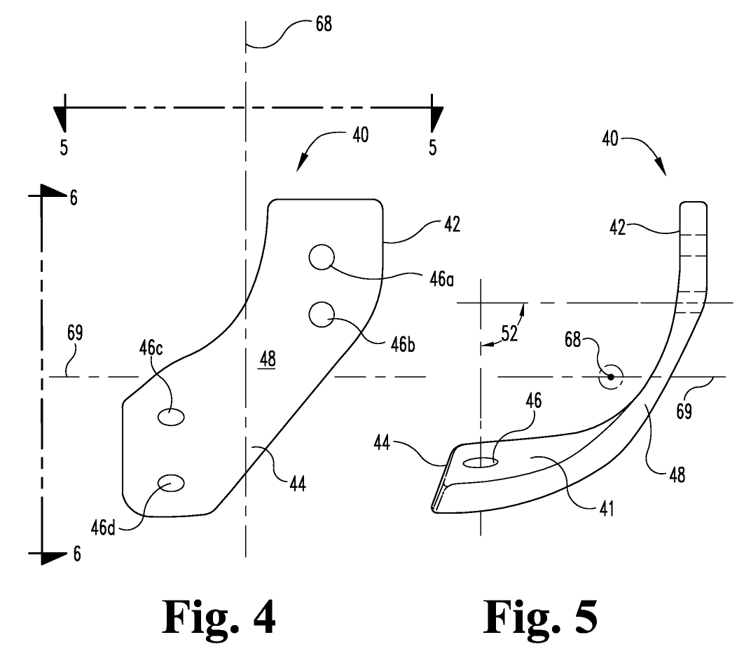

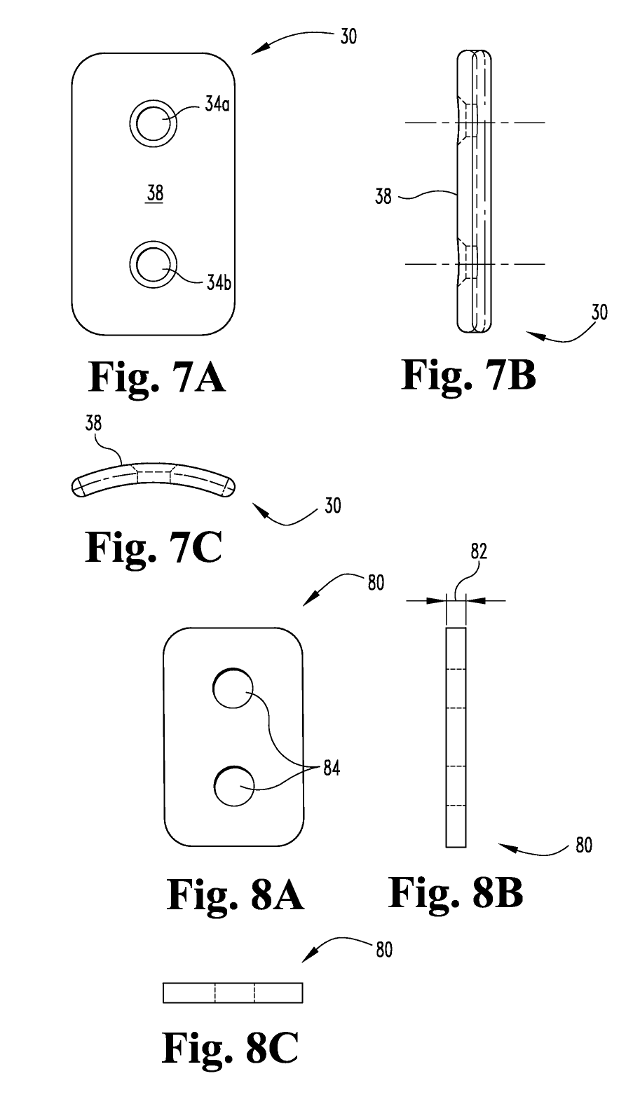

FIGS. 1, 2, and 3 depict the installation of the apparatus shown in FIGS. 4, 5, 6, 7 and 8. Referring first to FIG. 7, a primary plate 30 is shown according to one embodiment of the present invention. FIG. 7 presents a primary plate 30 shown in three orthogonally projected views. Plate 30 can be of relatively constant thickness and in some embodiments having a concave side 36 that is curved to improve its contact with the surface of a bone.

Plate 30 in some embodiments includes a pair of attachment holes 34a and 34b. Attachment holes 34a and 34b are adapted and configured to permit plate 30 to simultaneously interface with the bone on the concave side 36 and also interface with a secondary plate 40 on the convex side 38. In some embodiments, at least one of holes 34a or 34b extend through the thickness of plate 30 to permit the insertion of a bone anchor therethrough. In some embodiments, both holes 34a and 34b are through holes. In yet other embodiments, the other one of holes 34a or 34b are blind holes that are threaded to accept a fastener for securement of secondary plate 40.

The present invention contemplates producing primary plate 30 from any material suitable for inclusion in an animal body, and including as examples titanium and stainless steel.

FIGS. 4, 5, and 6 depict front and top views, respectively, of a secondary plate 40 according to one embodiment of the present invention. In one embodiment secondary plate 40 comprises an upper portion 42 and lower portion 44 interconnected by a central portion 48. Upper portion 42 includes one or more attachment holes 46a and 46b, and lower portion 44 includes one or more attachment holes 46c and 46d. Attachment holes 46a and 46b are adapted and configured for coupling of upper portion 42 to a primary plate 30, as will be described later. Attachment holes 46c and 46d are adapted and configured for coupling to a primary plate 34 that will be described later.

In one embodiment, secondary plate 40 is of a relatively constant thickness. However, the present invention also contemplates those embodiments in which the upper portion 42 has a first thickness, lower portion 44 has a second thickness, and the first and second thicknesses are different. This difference in thicknesses can accommodate particular bone geometry after rotation of the bone portions. Yet other embodiments of the present invention contemplate a secondary plate 40 having a variable thickness from upper portion 42 to lower portion 44.

As best seen in FIG. 5, secondary plate 40 includes a central portion 48 that is curved about an axis 68 that is substantially parallel to the longitudinal axis of a long bone. Therefore, the central axes of holes 46a and 46b are nonparallel to the central axes of attachment holes 46c and 46d. This curvature about axes 68 results in an included angle 52 (as projected into a plane perpendicular to axis 68 and parallel to axis 69). Secondary plate 40 thereby “wraps around” the longitudinal axis 68 of a long bone.

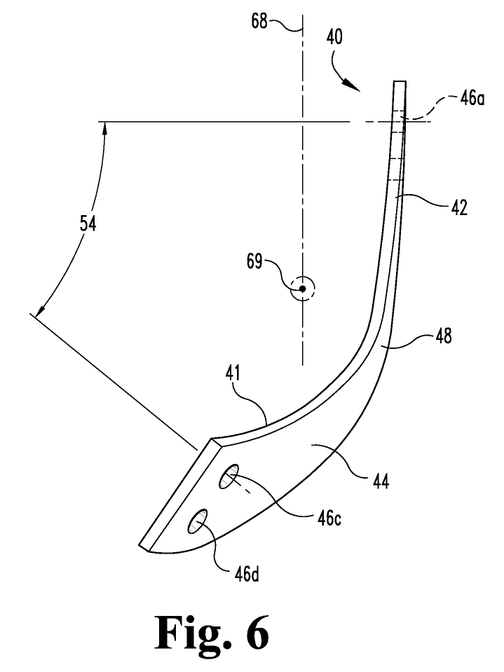

In some embodiments secondary plate 40 includes a central portion 48 that is curved about a lateral axis 69 that is substantially perpendicular to longitudinal axis 68. As best seen in FIG. 6, one of attachment holes 46c or 46d has a central axis that is nonparallel to the central axis of one of holes 46a or 46b. In some embodiments, there is an included angle 54 that indicates a degree of curvature of central portion 48 about lateral axis 69. Included angle 54 is shown as projected into a plane that is perpendicular to axis 69 and parallel to axis 68.

In yet other embodiments, secondary plate 40 is curved about axis 68 and also curved about axis 69. In these embodiments, this dual curvature is useful in providing a secure coupling between a pair of primary plates attached to a bone.

Although what has been shown and described are included angles 52 and 54 as established by the central axis of attachment holes, the present invention also contemplates those embodiments in which the angles 52 and 54 are defined from a first projection normal to the interior, primary plate-contacting surface 41 of upper portion 42 relative to second projection normal projected from the interior, primary plate-contacting surface 41 of lower portion 44.

Yet other embodiments of the present invention contemplate a kit 100 that includes a plurality of secondary plates with varying amounts of curvature. As one example, kit 100 includes a plurality of secondary plates such as plate 40 that are curved so as to have an included angle 52 ranging from 5 degrees to 90 degrees in predetermined increments, such as 5 degree increments. In yet other kits contemplated by the present invention each of the previously described incremental families (such as the family having an included angle 52 of 30 degrees) includes its own subfamily of secondary plates that have this same included angle 52, but are also curved to have an included angle 54 from 0 degrees to 20 degrees in predetermined increments, such as 2 degrees.

In some embodiments, a kit 100 further includes at least a pair of primary plates. In some embodiments, primary plates 30 and 32 are identical. In yet other embodiments, kit 100 includes a plurality of primary plates, each having a different thickness. In yet other families, the pair of attachment holes is moved 90 degrees, referring to FIG. 7, so that relative to the convex curvature of the bone-contacting side, the attachment holes are side-by-side laterally.

FIG. 8 depicts a shim or spacing device 80 according to one embodiment of the present invention. In one embodiment, spacing device 80 has a substantially uniform thickness 82. Further, spacing device 84 includes one or more through holes. In some embodiments, a spacing device 80 can be placed between a primary plate 30 and the bone undergoing surgery. In yet other embodiments, spacing device 80 is placed between the outer coupling surface 38 of plate 30 and the inner coupling surface 41 of secondary plate 40. Yet other embodiments of kit 100 include a plurality of spacing devices 80 from about 0.05 inches to about ¼ inch in predetermined increments such as increments of 0.05 inches.

FIGS. 1, 2, and 3 depict a method and use according to one embodiment of the present invention. Referring to FIG. 1, a pair of primary plates 30 and 34 are fastened to a bone. In one embodiment, plate 34 is identical to plate 30, although other embodiments of the present invention contemplate primary plates 30 and 34 that differ with regards to size, shape, thickness, material, surface finish, and the like.

Primary plates 30 and 34 are fastened to a bone 60 preferably by a bone anchor adapted and configured for secure attachment to the structure of a bone. The plates are installed along either side of a plane of the bone that is to be cut apart. Plates 30 and 34 are spaced apart by a sufficient distance at least to accommodate the thickness of the saw blade.

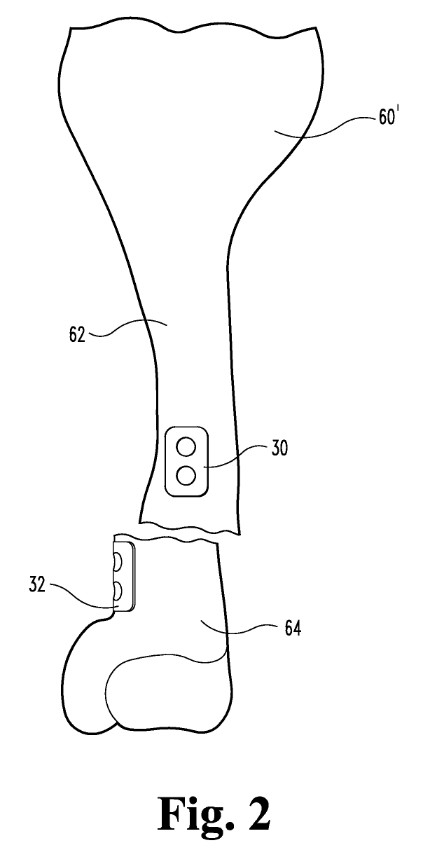

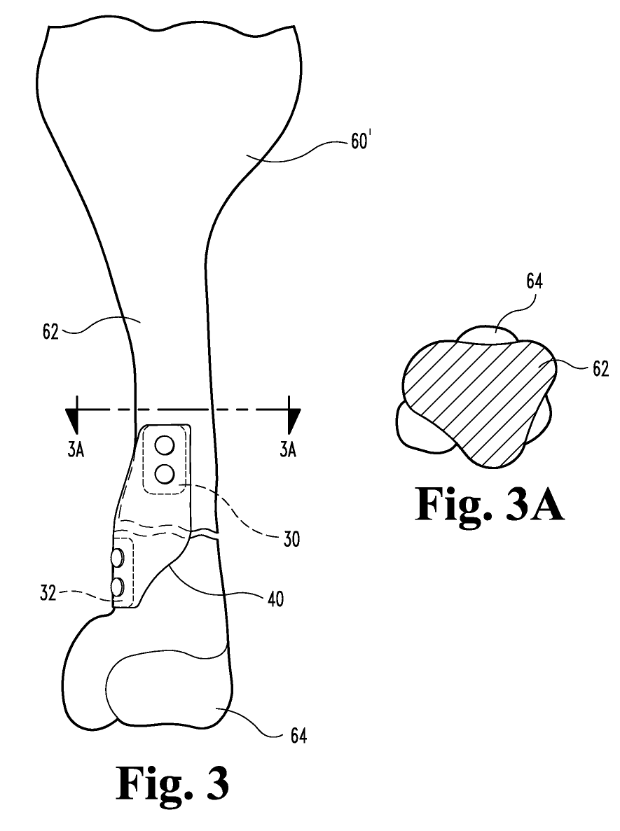

Referring to FIG. 2, after the cut the top primary plate 30 is attached to the upper portion of bone 60 and lower primary plate 30 is attached to the lower portion of the bone. The upper and lower portions of the bone are then rotated relative to each other to a new angular relationship. After the proper angular relationship is achieved, a secondary plate 40 is coupled so as to create a fixed relationship between primary plates 30 and 34 (as best seen in FIG. 3).

Various embodiments of the present invention contemplate different ways of coupling the secondary plate to the primary plate so as to fix the angular relationship of the upper and lower bone portions. Further, various embodiments of the present invention contemplate that the upper and lower primary plates 30 and 34 couple in the same manner to secondary plate 40, whereas other embodiments contemplate that plates 30 and 34 will be coupled to secondary plate 40 in different ways.

As one example, a primary plate is fastened to the bone by a bone anchor. A second fastener then couples the primary plate to the secondary plate. In such an embodiment, the primary plate would have one through hole and one threaded hole, and the secondary plate would have at least one through hole that aligns with the threaded hole of the primary plate.

As another example, the primary plate includes a through hole for fastening to the bone, and further includes a threaded stud that, after assembly, extends through a hole in the secondary plate, after which coupling is achieved by means of a nut. Preferably, the secondary plate includes a recessed portion surrounding the through hole to accommodate the thickness of the nut.

In yet another embodiment, the primary plate and secondary plate have their relative positions established by the coaction of an alignment pin extending from one of the plates and received within a blind hole of the other plate. in such embodiments, it is further possible to include a fastener that couples the primary plate and the second plate, such as a bolt in a threaded hole or a threaded shaft and a nut.

In yet other embodiments, the bone anchor that couples the primary plate to the bone is removed prior to installation of the secondary plate. The secondary plate includes a through hole that is aligned with the hole and the primary plate and the hole and the bone, and a bone anchor couples the secondary plate and primary plate to the bone proximate (and underneath) to the primary plate.

In yet other embodiments, the bone anchor that couples the primary plate to the bone is not firmly attached until the primary and secondary plates have been aligned. In yet other embodiments, the bone anchor is loosened to permit rotation of the primary plate about the anchor. With a loose primary plate, the primary plate and the secondary plate can be changed in their relative alignment, after which the primary plate can be tightened to the bone.

Yet other embodiments of the present invention contemplate mixing of the various attachment methods discussed herein.

While the inventions have been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. An apparatus for repairing a bone comprising:
a first plate adapted and configured for attachment to a long bone of an animal, said first plate having at least one hole for receiving a bone anchoring device therethrough;
a second plate adapted and configured for attachment to a long bone of an animal, said second plate having at least one hole for receiving a bone anchoring device therethrough;
a third plate having an upper portion for coupling to said first plate and a lower portion for coupling to said second plate, the upper portion having an angular offset relative to the lower portion in a first direction about a longitudinal axis of the bone.

2. The apparatus of claim 1 wherein the upper portion has a first thickness, the lower portion has a second thickness, and the first thickness is different than the second thickness.

3. The apparatus of claim 1 wherein the upper portion has an angular offset relative to the lower portion in a second direction about a second axis that is at least partly orthogonal to the longitudinal axis.

4. The apparatus of claim 1 wherein said third plate include a through hole for accepting a first fastener therethrough and said first plate includes a threaded hole for threadably accepting the first fastener

5. The apparatus of claim 4 wherein said third plate include a through hole for accepting a bone anchor therethrough, and said through hole of said third plate aligning with said at least one hole of said second plate, wherein said through hole and said at least one hole are capable of accepting the same bone anchor.

6. The apparatus of claim 1 which further comprises a spacer for insertion between said third plate and said first plate.

7. The apparatus of claim 1 wherein said first plate is curved about an axis that is generally parallel to the longitudinal axis of the bone.

8. The apparatus of claim 7 wherein said second plate is curved about the longitudinal axis of the bone.

9. The apparatus of claim 7 wherein the upper portion has an angular offset relative to the lower portion in a second direction about a second axis that is at least partly orthogonal to the longitudinal axis.

10. A method for repairing a bone, comprising:
providing two primary plates and a secondary plate;
fastening the first and second primary plates to the bone, one plate being fastened above the other plate, the plates being spaced apart from each other;
cutting through the bone between the plates;
rotating the two portions of the bone relative to each other to establish an angular relationship of the one bone portion relative to the other bone portion;
fastening the secondary plate to one of the first primary plate or the bone proximate to the first primary plate; and
fastening the secondary plate to one of the second primary plate or the bone proximate to the second primary plate.

11. The method of claim 10 wherein said fastening the secondary plate to one of the first primary plate or the bone proximate to the first primary plate is by fastening the secondary plate to the first primary plate.

12. The method of claim 11 wherein said fastening the secondary plate to one of the second primary plate or the bone proximate to the second primary plate is by fastening the secondary plate to the bone proximate to the second primary plate, the undersurface of the secondary plate being at least partly in contact with the second primary plate.

13. The method of claim 10 wherein said fastening the secondary plate to one of the first primary plate or the bone proximate to the first primary plate is by fastening the secondary plate to the bone proximate to the first primary plate, the undersurface of the secondary plate being at least partly in contact with the first primary plate.

14. The method of claim 13 wherein said fastening the secondary plate to one of the second primary plate or the bone proximate to the second primary plate is by fastening the secondary plate to the second primary plate.

15. An orthopedic kit for a bone, comprising:
at least two primary plates adapted and configured for attachment to a bone, each said primary plate having at least one hole for receiving a bone anchoring device therethrough; and
at least two secondary plates each adapted and configured for attachment to two primary plates, each said secondary plate having at least two holes for receiving fasteners therethrough for attachment of a said secondary plate to two said primary plates, each secondary plate being curved about a longitudinal axis of the bone, the curvature of one said secondary plate being different than the curvature of the other said secondary plate.

16. The kit of claim 15 which further comprises a plurality of spacers, each spacer having a different thickness than each other spacer, each spacer having at least one through hole.

17. The kit of claim 15 wherein the curvature is a first curvature, and each secondary plate having a second curvature about a second axis, the second axis being at least partly orthogonal to the longitudinal axis, the second curvature of one said secondary plate being different than the curvature of the other said secondary plate.

18. The kit of claim 17 wherein each said primary plate is curved about an axis that is generally parallel to the longitudinal axis of the bone.
